(19) United States
(12) Patent Application Publication
(10) Pub. No.: US 2008/0194919 A1
(43) Pub. Date: Aug. 14, 2008

(54) Method and apparatus for prediction and management of subjects and patients

(76) Inventors: Miranda Aref Farage, Cincinnati, OH (US); Philip Andrew Bowtell, Reading (GB)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/703,945

(22) Filed: Feb. 8, 2007

(51) Int. Cl. A61B 5/00

(52) U.S. Cl. 600/300

(57) ABSTRACT

A method for developing a questionnaire, the method comprising the steps of developing a database of “n” questions and answers relating to skin or health conditions; correlating the answeres to the “n” questions to actual skin or health conditions; and selecting from the “n” questions a set of “m” key question, wherein “m” is less than “n”, and wherein the “m” questions provides within a predetermined statistical probability a population of people having a particular skin or health condition. In one embodiment, the method can be used for determining among a population of people a subset of people who are likely to have atopic skin dermatitis. In one embodiment, the correlating step can be achieved by use of a computer running a statistical analysis program.

### FIELD OF THE INVENTION

The present invention relates to methods for determining a person's predisposition to certain skin, health or disease conditions.

### BACKGROUND OF THE INVENTION

People with atopic dermatitis are a subset of individuals that may have increased susceptibility to skin irritation or discomfort. Such people are likely to -have clinically meaningful skin complaints. The portion of the population having clinically meaningful skin complaints can be important for products research involving skin care products. A high proportion of product-use related skin effects are transient, and consumers are able to reuse the product at a later time without incident. But for some people, product related complaints are due to their skin's susceptibility to irritation.

A quick method to distinguish between persons or groups of people who are predisposed to skin complaints from use of skin care compositions and consumer products would be useful in a variety of fields. For example, post-market surveillance of consumer products could identify consumers who experience product-related skin complaints. Such complaints could require follow up to determine the cause of the problem. By having a quick method to distinguish between those who are simply experiencing transient skin problems and those having clinical dermatitis, such complaints can be dealt with effectively. Effective selection can be beneficial in recruiting individuals to test new products, or to diagnose those who have used consumer products.

It would be desirable to have a method for determining which people are likely to have clinically diagnosable skin, health or disease conditions and which people have transient skin complaints or distinguish between normal and clinically diagnosed health or skin conditions.

Further, it would be desirable to have a method for determining which people are likely to have topic dermatitis and which people have skin complaints unrelated to atopy.

### SUMMARY OF THE INVENTION

A method for developing a questionnaire, the method comprising the steps of developing a database of “n” questions and answers relating to skin or health conditions; correlating the answers to the “n” questions to actual skin or health conditions; and selecting from the “n” questions a set of “m” key question, wherein “m” is less than “n”, and wherein the “m” questions provides within a predetermined statistical probability a population of people having a particular skin or health condition. In one embodiment, the method can be used for determining among a population of people a subset of people who are likely to have atopic skin dermatitis or other skin or health conditions. In one embodiment, the correlating step can be achieved by use of a computer running a statistical analysis program.

### DETAILED DESCRIPTION OF THE INVENTION

The invention is a fast, simple method for determining among persons or groups of people who is likely to have clinically diagnosable skin, health or diseased conditions, for example, atopic skin dermatitis. The method can be used to distinguish between problem (or clinically diseased) related population versus normal population or patients.

The method involves reducing the number of questions necessary to make a probabilistic determination of clinically diagnosable skin, health or diseased conditions. To reduce the number of questions to one, two, or a few key questions, the investigator relies on a database of questions asked and answered in the past. Using a database of past questions and answers related to skin health conditions, statistical analysis can be performed to derive the minimum number of key questions necessary to predict with a high degree of accuracy a consumer or patient's diagnosis. For example, the contents of a database of questions, answers, and clinical diagnosis of a relatively large population of people can be correlated by use of a commercial statistics program, such as ANSWERTREE®, (available from SPSS, Inc. Chicago, Ill.). In one embodiment, a probability limit can be specified, and the statistic program can correlate questions and answers to find the minimum combination of key questions to deliver correct diagnoses within the probability limit. The statistical program can also determine the order of asking of key questions. The resulting questionnaire can be much more simple and quick to administer, and can be administered by either a professional or non-professionals where warranted.

In one embodiment, the method of developing key questions, and the key questions themselves, can be utilized in post-market or post-use surveillance of consumer or medical products. Consumers and/or patients can be tracked post-market to track skin complaints, health problems, or disease conditions. Answers provided can be used to determine individuals within a population that show unanticipated trends in skin and/or heath conditions. Questions and answers can be used to diagnose medical conditions by a medical professional, or they can be used by consumer products manufacturers to determine post-use experiences of users of consumer products.

In one embodiment, the method of developing key questions, and the key questions themselves, can be utilized to recruit individuals to test consumer products and/or participate in clinical studies. By use of the key questions developed by the method of the invention, individuals can be identified who can participate in tests, as well as those who should not.

In one embodiment, the method of developing key questions, and the key questions themselves, can be utilized to screen patients for medical procedures. A dermatologist or other medical professional, for example, could administer a short questionnaire to quickly segregate a population of patients or potential patients with specific skin or health conditions.

The questions in the questionnaire rely on people to answer based on their self-perceived skin or health conditions and product use habits. The two-step process was derived by analyzing a variety of questions by statistical analysis, and in a small base study of 50 people has shown itself to be 90% reliable by correctly identifying 22 of 25 people with atopic dermatitis and 23 of 25 people without atopic dermatitis.

The invention is described below in one embodiment in which a database of hundreds of questions and answers related to atopic dermatitis was analyzed and processed by ANSWERTREE® software to derive three key questions to be delivered in two steps. The key questions and method are discussed below in the context of determining from a population of people those individuals who have perceived skin sensitivity, and in particular, atopic dermatitis. Of course, as discussed above, similar key questions could be utilized to determine other skin or health conditions.

For each of the questions discussed herein, it is noted that the questions need not be word-for-word the same as that disclosed. For each question disclosed and claimed herein, it is intended that the method include similarly-worded questions, or different questions which elicit the same kind of information which can be used to separate people based on perceived skin sensitivity or health conditions. Likewise, for the answer choices disclosed and claimed herein, the invention is not intended to be limited to these answers word-for-word, but can be any answers which elicit the same kind of information which can be used to separate people based on perceived skin or health sensitivity.

Step one of the illustrated two-step process is to ask the person (or people, if more than one person is being evaluated) the question: “How would you describe your skin?” The person (or people) is given a choice of answers, including “very sensitive”, “moderately sensitive”, “slightly sensitive” and “not sensitive”. The answer(s) is/are recorded, and the person (or people) then proceeds to Step Two. Answers of “very sensitive”, “moderately sensitive”, are considered potentially predictive of atopic dermatitis. Current testing of a group of 50 people shows on the basis of this question 24/50 people were classified as “likely to have atopic dermatitis” with 21/24 being correctly classified. Of those individuals who answered “slightly sensitive” or “not sensitive”, current testing of the same group of 50 people shows 26/50 classified as “unlikely to have atopic dermatitis” with 21/26 being correctly classified.

Step two is to ask one of two questions, with the question asked being dependant on the answer to the question in Step One. Of those classified in Step One as “likely to have atopic dermatitis”, then the question asked in Step Two is: “Do you shop for skin care products that advertise ‘save for sensitive skin’ or ‘hypoallergenic’?” The answer choices for this second question are “yes” and “no”. Those in this group who answered “yes” to this second question are classified as “likely to have atopic dermatitis”, with current testing showing the correlation to be 19/21, and when combined with the first question in Step One the method yields accurate predictability of a person having diagnosable dermatitis of about 21/24 or about 88%.

Of those classified in Step One as “unlikely to have atopic dermatitis”, the question asked in Step Two is: “Have alpha hydroxy acids ever caused your skin to be irritated or uncomfortable?” The answer choices for this second question are “frequently”, “don't use”, “sometimes”, and “never”. Those in this group who answered “frequently” or “don't use” to this second question are classified as very” likely to have atopic dermatitis”, with current testing showing the correlation to be 100%, and when combined with those classified as “likely to have to have atopic dermatitis” from Step One, the method yields accurate predictability of a person having atopic dermatitis of 24/26 or about 92%.

Therefore, the algorithm utilized and illustrated above with respect two the two-step process involving three questions, one can predict the likelihood of a person having clinically diagnosable dermatitis, specifically atopic dermatitis to a confidence level of greater than 90%.

The questions may be refined for other purposes, or for the same purpose but with different groups of people. Perceived skin sensitivity and associated behaviors may not be a predictive among certain portions of the population, or for some individuals. Therefore, in addition to the questions discussed above, other questions may need to be asked. For example, some preliminary questions can help ascertain the meaning of the questions, so that the answers are more predictive and accurate.

The method of the present invention can be utilized to diagnose skin health conditions or determine post-use experience information for other skin conditions such as acne, dandruff, gum disease, and the like. For each skin condition the type and number of questions can be different, tailored to the particular skin condition, and drawn from a relevant database of questions/answers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for determining among a population of people a subset of people who are likely to have atopic skin dermatitis, said method comprising the steps of:
a. providing a first plurality of people;
b. asking each of said first plurality of people a first question: “How would you describe your skin?”;
c. having each of said first plurality of people answer said first question by selecting one answer from the group consisting of, “very sensitive”, “moderately sensitive”, “slightly sensitive” and “not sensitive”;
d. asking a second plurality of people consisting of those of said first plurality of people who answered “very sensitive” or “moderately sensitive” a second question: “Do you shop for skin care products that advertise ‘safe for sensitive skin’ or ‘hypoallergenic’?”;
e. having each of said second plurality of people answer said second question by selecting one answer from the group consisting of, “yes” and “no”;
f. asking a third plurality of people consisting of those of said first plurality of people who answered “slightly sensitive” and “not sensitive”, a third question:
“Have alpha hydroxy acids ever caused your skin to be irritated or uncomfortable?”;
g. having each of said third plurality of people answer said third question by selecting one answer from the group consisting of, “frequently”, “don't use”, “sometimes”, and “never”; and
h. selecting a fourth plurality of people, said fourth plurality of people being those who answered “yes” to said second question, those who answered “frequently” or “don't use” to said third question, wherein said fourth plurality of people are people likely to have atopic skin dermatitis.

2. The method of claim 1, wherein said first plurality of people includes people in a post-market survey of consumer product use.

3. The method of claim 1, wherein said first plurality of people includes people in a post-use survey of medical product use.

4. The method of claim 1, wherein said first plurality of people includes people being screened for recruitment for participation in evaluation of consumer products.

5. The method of claim 1, wherein said first plurality of people includes people being screened for recruitment for participation in evaluation of medical products.

6. A method for developing a questionnaire, said method comprising the steps of:
a. developing a database of “n” questions and answers relating to skin or health conditions;
b. correlating said answers to said “n” questions to actual skin or health conditions;
and
c. selecting from said “n” questions a set of “m” key questions, wherein “m” is less than “n”, and wherein said “m” questions provides within a predetermined statistical probability a population of people having a particular skin or health condition.

7. The method of claim 6, wherein “n” is greater than 10 and “m” is less than or equal to 3.

8. The method of claim 6, wherein “n” is greater than 100 and “m” is less than or equal to 5.

9. The method of claim 6, wherein “n” is greater than 1000 and “m” is less than or equal to 10.

10. The method of claim 6, wherein said skin or health condition is dermatitis.

11. The method of claim 6, wherein said skin or health condition is selected from dandruff, acne, and oral health conditions such as gingivitis and other gum diseases.

12. A method for developing a questionnaire, said method comprising the steps of:
a. developing and providing a database of “n” questions and answers relating to skin or health conditions;
b. providing a correlation between said “n” questions and answers to actual skin or health conditions;
c. providing a computer capable of running a computer program;
d. providing a statistical analysis computer program capable of correlating said questions and answers to said “n” questions;
e. running said statistical analysis computer program on said computer to correlate said questions and answers to said “n” questions and said actual skin or health conditions, and to select from said “n” questions a set of “m” key questions, wherein “m” is less than “n”; and
f. wherein said “m” questions provides within a predetermined statistical probability a population of people having said actual skin or health condition.

13. The method of claim 12, wherein “n” is greater than 10 and “m” is less than or equal to 3.

14. The method of claim 12, wherein “n” is greater than 100 and “m” is less than or equal to 5.

15. The method of claim 12, wherein “n” is greater than 1000 and “m” is less than or equal to 10.

16. The method of claim 12, wherein said skin or health condition is dermatitis.

17. The method of claim 12, wherein said skin or health condition is dandruff.

18. The method of claim 12, wherein said statistical analysis computer program is ANSWERTREE® available from SPSS, Inc.
